(19) United States
(12) Patent Application Publication
(10) Pub. No.: US 2025/0256052 A1
(43) Pub. Date: Aug. 14, 2025

(54) Wearable Pressure Applying Device

(71) Applicant: Dwight Joel Lee Koyner, Weston, CT (US)

(72) Inventor: Dwight Joel Lee Koyner, Weston, CT (US)

(21) Appl. No.: 18/438,425

(22) Filed: Feb. 10, 2024

(51) Int. Cl. A61M 21/00; A61H 7/00

(52) U.S. Cl. CPC A61M 21/00; A61H 7/001; A61H 2201/0192; A61H 2201/1215; A61H 2201/1621; A61H 2201/165; A61H 2201/168; A61H 2205/08; A61M 2021/0022; A61M 2205/10; A61M 2210/10

(57) ABSTRACT

The invention pertains to a wearable pressure-applying device designed for user comfort and convenience. The device features multiple securing mechanisms for attachment to the user, including adaptable closures such as zippers. A manual or mechanical engagement system allows the user to adjust the constriction of the device, applying pressure as desired. Notably, the closure mechanisms do not extend across the entire vertical length of the wearable device, enhancing flexibility and comfort. The constriction mechanism employs either threaded or stringed lacing methods, with separable connections for user attachment and removal. An ergonomically contoured handle, easily attachable and detachable via a coupler, enhances usability. Additionally, a cord lock mechanism ensures precise pressure control, with a quick-release feature for instant pressure relief. Modular attachment options for both mechanical and cord-lock mechanisms offer versatility. Furthermore, advanced versions of the device may incorporate battery-powered or sensor-driven pressure application capabilities, with potential for wireless control.

### FIELD OF THE DISCLOSURE

The present invention relates to systems to contract a garment around an individual so as to squeeze them.

### BACKGROUND ART

Deep pressure is a type of surface pressure exerted through swaddling, holding, or a firm hug. Deep pressure therapy (DPT) has been shown to produce a calming sensation in individuals with sensory processing disorders including Autism Spectrum Disorder (ASD), Attention-Deficit/Hyperactivity Disorder (ADHD), and Alzheimer's. See Faulcon, Kaitlin, “The Effect of Deep Pressure Therapy on the Health of Individuals With Developmental Disorders” (2020). Capstone Showcase. 39. See Lane, Shelly J, and Stacey Reynolds. “Sensory Over-Responsivity as an Added Dimension in ADHD” (2019). Frontiers in integrative neuroscience vol. 13 40. 6. Many devices that seek to use deep pressure therapy lack ease of use for a user with sensory disorders, for example, autism. Others are bulky or not portable. Still, others have difficulty being cleaned by washing machines. Further, other Deep Pressure Therapy devices serve only consumers who have sensory processing disorders, thereby functioning as a label of said sensory processing disorder, as well as neglecting to adapt the hugging sensation of deep pressure therapy devices to a broader range of users to meet a wider scope of needs. It is known that there are many health benefits to hugs, such as reduced heart rates, improved mood, and lower blood pressure. Research also shows that hugs can reduce levels of the stress hormone cortisol.

### REFERENCES

Cohen, S., Janicki-Deverts, D., Turner, R. B., & Doyle, W. J. “Does Hugging Provide Stress-Buffering Social Support? A Study of Susceptibility to Upper Respiratory Infection and Illness” (2015). Psychological Science, 26(2), 135-147.

### SUMMARY OF THE INVENTION

This summary section provides an overview of the some of the inventive aspects related to the device, method and apparatus as outlined in the description. It is not a comprehensive review of the invention and is not meant to restrict the scope to anything beyond what is described and claimed in this summary.

The present application discloses a wearable pressure-applying device for both therapeutic and personal use. The wearable pressure-applying device may simulate a feeling of being held or contained. The wearable pressure-applying device may include an application apparatus of strings or laces substantially embedded or attached to the wearable device at a number of positions to be tightened to provide a compression force to the user when the user is wearing the device, for example, to simulate a feeling of being hugged. The tightening mechanism may be of mechanical, manual, or other suitable means, wherein the wearable pressure-applying device can modularly attach a number of different tightening mechanisms interconnected through separable connections. Such a design enables the wearable pressure-applying device to more easily be cleaned and enables the pressure application mechanism to best suit the abilities of various users with distinct needs and differing abilities. In some embodiments, the manual means of the tightening mechanism may be designed as to accommodate a range and fineness of motion appropriate to individuals with disabilities, such as autism, where the said design may only require more basic motor skills to activate. In some embodiments, the means of the mechanical tightening mechanism may be through the use of a motor, for example, to pull the application apparatus. Further, in other embodiments, the mechanical mechanism may include a plurality of different sensors to sense the physiological parameters of the user, such as a heart rate sensor, the environmental parameters of the user's environment, such as a decibel meter, and a dynamics sensor of the wearable pressure-applying device motion, such as an accelerometer. These sensors may be configured to initiate the pressure-applying mechanism if certain conditions or thresholds are reached. In some embodiments, the means of the mechanical tightening mechanism may be controlled wirelessly, for example, from the phone of the caregiver of a user. This design may allow a caregiver to apply pressure to the wearer, as to calm them with deep pressure therapy, or the sensation of a hug, for example for individuals with autism who may not be emotionally capable of manually applying deep pressure therapy for example, when experiencing increased anxiety.

The wearable pressure-applying device may include a plurality of mechanisms for bounding itself to an individual. One said mechanism may be a clasp locker. Such a design enables the wearable pressure-applying device to be adaptable in size, for example, to fit variously sized users. Other mechanisms may be hook-and-loop fasteners, buttons, or other suitable means. Any number of these mechanisms may be used in combination, as to enable multiple different manual ways of securing or fitting the wearable pressure-applying device to the user. Further, the application of any of these mechanisms may also apply an initial and regular pressure to the user which may enable micro-adjustments to the pressure, for example, a clasp locker. Still, any number of said mechanisms may detachably connect sides of the wearable pressure-applying device or may be confined entirely within a portion of the wearable device.

Through the use of multiple different mechanisms to apply pressure, in both mechanical and/or manual means of tightening of the application apparatus, as well as a number of different dual-functioning securing and manual pressure-applying instruments, the present invention adapts itself to enable users of differing fine motor skills and self-awareness to don the wearable pressure-applying device equally, as well as enable all of these users to have multiple different systems disposed to apply pressure, being of differing power, extent, and span. This design allows users of various needs to experience an initial and constant pressure during the time they are donning the wearable pressure-applying device, as well as the flexibility to have adjusted the sensation of pressure while wearing said device, for example, in moments of particular distress.

This disclosure will now provide a more detailed and specific description that will refer to the accompanying drawings. The drawings and specific descriptions of the drawings, as well as any specific or alternative embodiments discussed, are intended to be read in conjunction with the entirety of this disclosure. The Wearable Pressure Applying Device may, however, be embodied in many different forms and should not be construed as being limited to the embodiments set forth herein; rather, these embodiments are provided by way of illustration only and so that this disclosure will be thorough, complete and fully convey understanding to those skilled in the art.

### BRIEF DESCRIPTION OF THE DRAWINGS

The novel features believed to define the illustrative embodiments are detailed in the appended claims. To fully comprehend these embodiments, along with their preferred usage, objectives, and detailed descriptions, one should refer to the comprehensive description of one or more examples of these embodiments, as provided in this disclosure. This understanding is further enhanced when considered alongside the accompanying drawings, wherein:

FIG. 1 illustrates a front exterior view of an embodiment of a garment that can be fitted with manual and/or mechanical engagement apparatuses.

FIG. 2 illustrates a front exterior view of an embodiment of a garment fitted with a mechanical engagement apparatus.

FIG. 3 illustrates a front exterior half view of an embodiment of a garment fitted with a manual engagement apparatus.

FIG. 4 illustrates an isometric interior view of an embodiment of a mechanical engagement apparatus.

FIG. 5 illustrates an exterior view of an embodiment of a mechanical engagement apparatus.

FIG. 6 illustrates a back exterior view of a user donning an embodiment of a garment fitted with a manual engagement apparatus.

FIG. 7 illustrates a side exterior view of a garment fitted with one particular embodiment of an application apparatus.

FIG. 8 illustrates a side exterior view of a garment fitted with another particular embodiment of an application apparatus.

### DETAILED DESCRIPTION OF PREFERRED EMBODIMENTS

The detailed description of the preferred embodiments of this invention is presented here, with references to the accompanying drawings. The specific terms and words used in both the description and the claims of this invention should not be confined to their ordinary or dictionary definitions. Instead, their interpretation should align with the meanings and concepts relevant to the invention, reflecting the inventor(s)' ability to define terms uniquely to best convey the invention.

It should be noted that the embodiment of the invention illustrated and discussed in this document represents a preferred example, and does not intent to restrict the technical essence or boundaries of the invention. Therefore, it is important to acknowledge that various alterations and adaptations can be made to the invention, which are still within its spirit and scope.

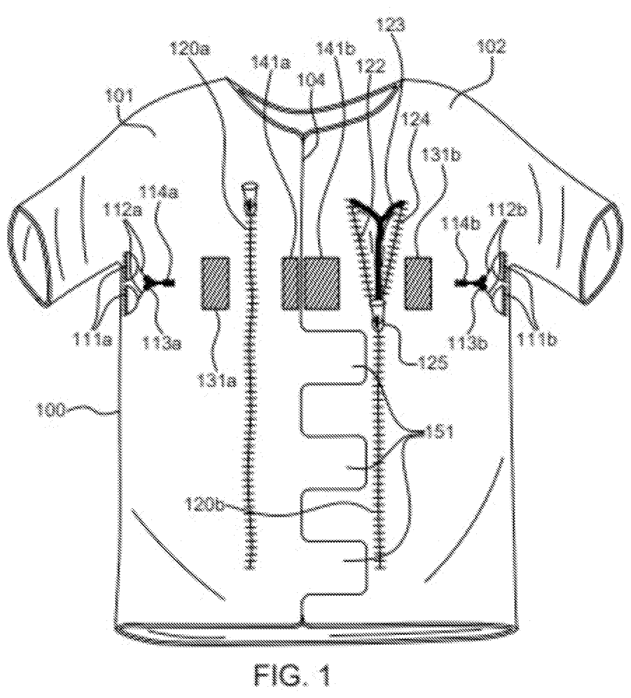

The present invention is directed to wearable pressure applying device. FIG. 1 is a front exterior view of an example embodiment of the present invention 100. The wearable pressure applying device 100 comprises of sides 101 and 102, which join into a single-piece assembly. When fitted to an individual, the lateral projections 151 form ‘tabs’ that may be used to adjust the fit of the garment to the individual wherein said individual may use said tabs to connect sides 101 and 102 to secure themself to the system, whereby said tabs may affix to side 102 at various lateral locations disposed to adjust the width of the system. In the preferred embodiments, the connection method is hook-and-loops, however, other embodiments may use buttons, zippers, or any other suitable means. The initial fitting of the garment to an individual may apply an initial pressure or may be used to alter the width of the garment, for example, to fit variously sized individuals or individuals who may change in size. Clasp locks 120a and 120b allow individuals to apply slight adjustments to the pressure of the garment, or as to alter the width of the garment, for example, to fit variously sized individuals or individuals who may change in size. Teeth 122 are separated from corresponding teeth 124 by channel 123. Channel 123 extends lengthwise throughout the entire length of 120b. Slider 125 adjoins 122 and 124 into 120b thereby folding channel 123 onto garment 100. Hook-and-loop patches 131a, 131b, 141a, and 141b allow the modular engagement and disengagement of the manual and mechanical engagement apparatus'. In alternative embodiments, the use of buttons, clasp locks, or any other suitable means may secure the engagement apparatuses to wearable device 100.

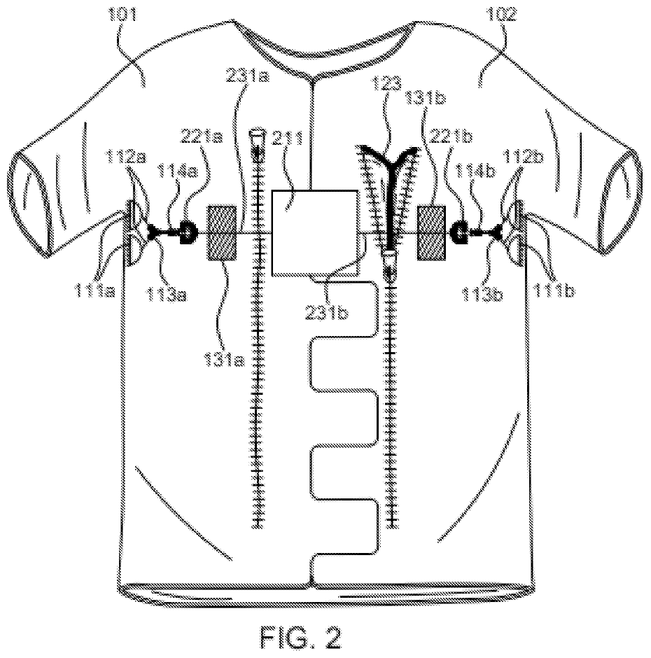

FIG. 2 is a front exterior view of an example embodiment of the present invention 100. In this embodiment, the mechanical pressure applying apparatus 211 is engaged to the device 100. In the example embodiment, hook-and-loop fasteners covering the rear of the modular mechanical engagement apparatus and respectively face portions of sides 101 and 102 to secure 211 to 100. In this embodiment, 211 may pull on strings 231a and 231b in the inward direction. Preferably, strings 231a and 231b have some substantial elasticity to give lead to the system. When strings 231a and 231b are pulled inwardly, connector 113a and 113b pulls on string segments 112a and 112b which connect to load distributors 111a and 111b respectively. Load distributors 111a and 111b distribute the pull force of 231a and 231b across a substantial part or parts of an individual's torso, such as the chest. The load distributor is highly preferable to the present invention as to better simulate the enclosing of a hug, as well as to apply pressure across a greater length of an individual's torso. In the example embodiment, connectors 113a and 113b and string 231a and 231b are interrupted by coupler mechanisms consisting of 114a and 221a as well as 114b and 221b. In the example embodiment, the coupler mechanism is composed of two parts, which can be engaged by insertion, and disengaged by tugging. In other embodiments, a snap, buckle, hooks, or other means of fastening may interrupt strings 231a and 231b and connectors 113a and 113b.

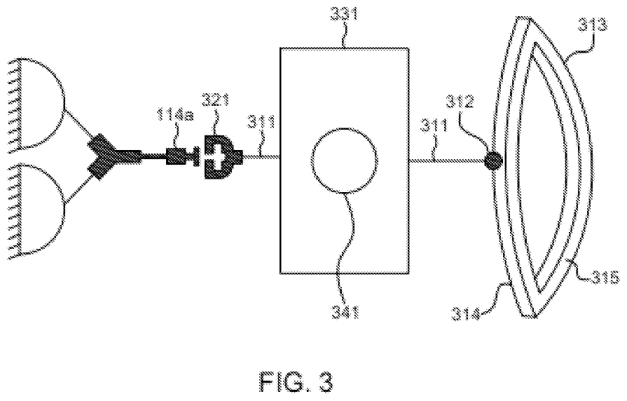

FIG. 3 is a full view of the front of an example embodiment of a manual engagement apparatus. Handle 313 is composed of Extrusions 314 and 315 which are separated by a substantially obtuse angle to conform to the hand of an individual, for example when making a fist. An individual may wrap their fingers around extrusion 315, for example, to help them engage the manual engagement apparatus. String 311 connects to handle extrusion 314 at connection 312, as well as to coupler part 321 corresponding to coupler part 114a. String 311 goes through cord lock mechanism 331. Preferably, the present invention includes cord lock mechanism 331 as part of the manual engagement apparatus. Cord lock mechanism 331 allows an individual to pull on handle 313, as to apply a force to string 311 in a direction substantially perpendicular to the front side of garment 100 running lengthwise, and release, thereby folding some amount of garment side 101 onto itself, applying pressure, and allowing said individual to then release handle 313 and have no substantial change to pressure sensation. Cord lock 331 achieves this by only allowing uni-directional movement of string 311, namely, in the inwards direction. In the present embodiment, an individual may disengage the cord lock to enable bi-directional movement of string 311 by pressing button 341. In other embodiments, any suitable mechanical, or electronic means may be used to engage or disengage cord lock mechanism 331. In the present embodiment, cord lock mechanism 331 is fitted to garment side 101 by use of hook-and-loop fasteners as depicted in FIG. 1 as 131a, as well as corresponding hook-and-loop fasteners on the corresponding parts of the back of cord lock 331, however, in other embodiments, and suitable means of fastening may be used.

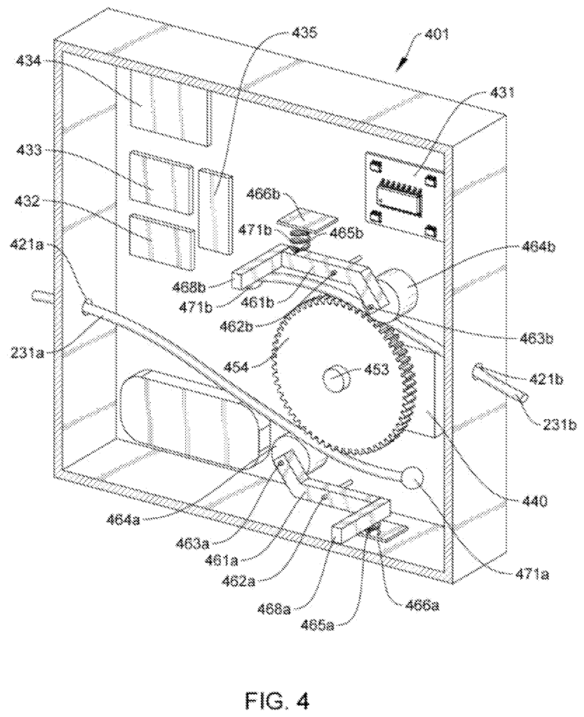

FIG. 4 is a front interior view of an example embodiment of mechanical engagement apparatus 211. In the example embodiment of the mechanical engagement apparatus, the method by which motor 440 applies pressure to the individual is through the pulling of strings 231a and 231b wherein the method of said mechanism in the present embodiment consists of the following: bars 461a and 461b are connected to apparatus enclosure 401 through axle/pivot 462a and 462b respectively. Connected to bars 461a and 461b are pulleys 464a and 464b through axle 463a and 463b respectively. In the example embodiment, springs 467a and 467b connect to bars 511a and 511b through connection points 465a and 465b respectively. Connection points 465a and 465b sit on the opposing side of pivots 462a and 462b from pulleys 464a and 464b. Springs 467a and 467b are in compression, and also connect to points 466a and 466b which themselves are fixed to encasement 401, thereby creating a clockwise force on bars 461a and 461b through pivots 462a and 462b. In alternative embodiments, any appropriate means to create such force may be used, for example, an elastic band. In the present embodiment, the clockwise force created by elastic bands 467a and 467b force pulleys 464a and 464b into contact with strings 231a and 231b at points 469a and 469b, thereby pushing said strings into contact with gear 452's teeth 454. When DC motor 440 rotates gear 452 counterclockwise through axle 453, strings 231a and 231b are pulled in the inwards direction, thereby applying pressure to the user. In the present embodiment, strings 231a and 231b are fixed to encasement 401 at points 571a and 571b respectively. In alternative embodiments, mechanisms to prevent the clumping of strings 231a and 231b as they are compiled may be used. Vertical extensions 568a and 568b sit on the opposite side of pivots 462a and 462b from pulleys 464a and 464b. Quick-release vertical extensions 468a and 468b extrude through encasement 401 to the exterior, in a manner that is accessible to an individual. When an individual applies an upward force to quick-release extension 468a, or downward force to quick-release extension 468b, bars 461a and 461b respectively rotate counterclockwise, thereby disengaging pulleys 464a and 464b from pushing strings 231a and 231b against gear 452, enabling free movement of strings 231a and 231b. Geared DC motor 440 is controlled by microcontroller 431. In the example embodiment, battery 433 provides power to microcontroller 431, motor 440, and sensors 432, 433, 434, and 435, however, in alternative embodiments, power may be generated by kinetic movement of individual, for example, piezoelectric generators or environmental conditions, for example, solar energy. In further alternative embodiments, power may be delivered through connection to an outlet or other external power source. Another way is wirelessly through a mobile device such as a smartphone, which may connect to microcontroller 431 by any suitable means, such as Bluetooth. The mobile device may be controlled by a parent, caretaker, or the individual donning the garment. The mobile device user may manually set the applied pressure remotely, for example, through the use of a slider. A caretaker may also change the mode of the mechanical engagement apparatus to automatic, wherein a plurality of sensor data is used as an input to a function which outputs the needed pressure towards the individual. In this embodiment, sensors 432, 433, 434, and 435 are a heart rate sensor, an accelerometer, a decibel meter, and a Galvanic Skin Response sensor respectively. Sensors may measure physiological data of the individual, such as their heart rate, environmental conditions, such as the noise level, as well as physical conditions, such as the individual's acceleration or other means of tracking common causes, or symptoms of distress. The data from these sensors is parsed by the microcontroller 431, and an output representing the needed pressure for the individual will be produced, and motor 440 will be controlled appropriately. The weight of each sensor's data can be set by caretakers or individuals wirelessly controlling apparatus 211 as to better adapt pressure activation to an individual's scenario, for example individuals who rock back and forth when in need of deep pressure therapy may set the weighting for the accelerometer as high.

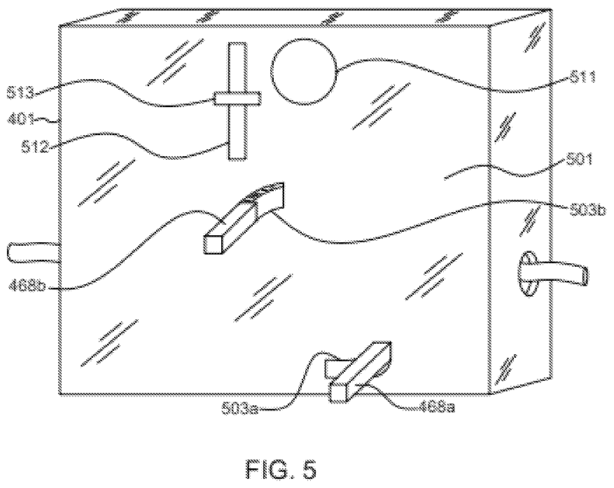

FIG. 5 is an exterior front view of an example embodiment of the mechanical mechanism used in the mechanical engagement apparatus as depicted in FIG. 4. Encasement 501 encases internals described in FIG. 4. Encasement 501 has slots 503a and 503b to allow for the movement of vertical extrusions 468a and 468b, as to allow for the quick release mechanism described in FIG. 4 to function properly. The degree of the applied pressure to the individual can be set in multiple ways. One such way is by the use of the manual slider 412, which protrudes to the exterior of encasing shell 501. The height of the slider knob 413 determines the applied pressure. The mechanical engagement apparatus engaged can manually turn on and off through the use of on/off switch 511. In the example embodiment, switch 511 protrudes through shell 501 to the exterior.

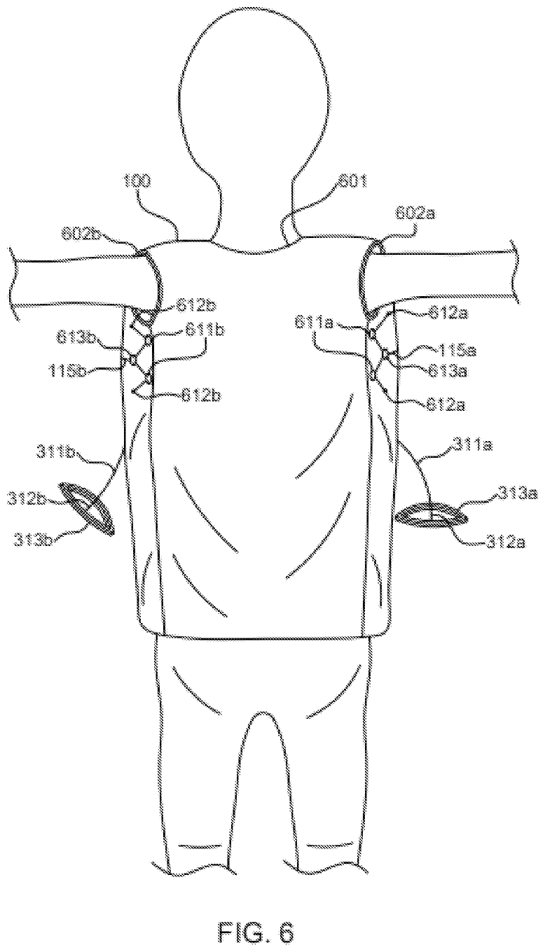

FIG. 6 is a back exterior view of an embodiment of a garment. In the example embodiment, the garment is fitted with a manual engagement apparatus. The manual engagement apparatus is connected to the mechanism adapted to constrict the device. Strings 115a and 115b connect to couplers not depicted, which connect to the manual pressure application apparatus. In the present embodiment, strings 115a and 115b split at points 613a and 613b respectively. These strings all diverge in substantially opposite directions and connect to grommets 611a and 611b. Said strings are laced through the grommet and return to a fixed point 612a and 612b respectively which are substantially vertically aligned with split points 613a and 613b. In this configuration, when stings 115a or 115b are pulled in the inwards direction, they bring the vertical line formed by points 611a or 611b to meet the other vertical line formed by points 612a and 613a, and 612b and 613b respectively, thereby constricting the device, and applying a sensation of pressure to the user.

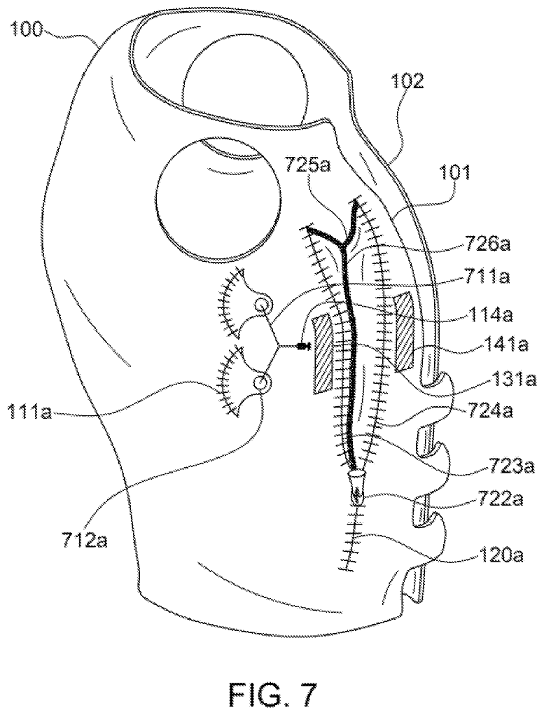

FIG. 7 is a side exterior view of one embodiment of a garment. In this embodiment, channel 726a is formed by sides 723a and 724a as well as seam 725a. Rows of mounted teeth 723a and 724a meet at slider 722a to form interlocking seam 120a. In the present embodiment, rows of teeth 723a and 724a are of a substantial distance apart as to enable the conjoining of these rows to contract the device 100 and reduce in the width device 100. Zipper mechanism composing of 722a, 723a, 724a, 725a, 726a, and 120a may be aesthetically pleasing or otherwise interesting to look at. In the present embodiment, coupler part 114a connects to multiple force spreaders through strings in a substantially symmetrical way across a lateral axis. Strings connects to the force spreader at point 712a. These points may be designed in a way to enable the detachment and reattachment of strings to force spreaders. In the present embodiment, force spreaders compose of base 111a and connection 711a. Base 111a is designed to span a substantial vertical distance outwards from the connection points 712a, whereas connection 711a serves to physically connect and transfer force from force spreader into the device 100 across a vertical area which substantially covers the vertical span of the torso of some user. In other embodiments, a force-spreading mechanism may be discretely embedded into the device. In alternative embodiments, any appropriate means of spreading the force over some larger distance may be used.

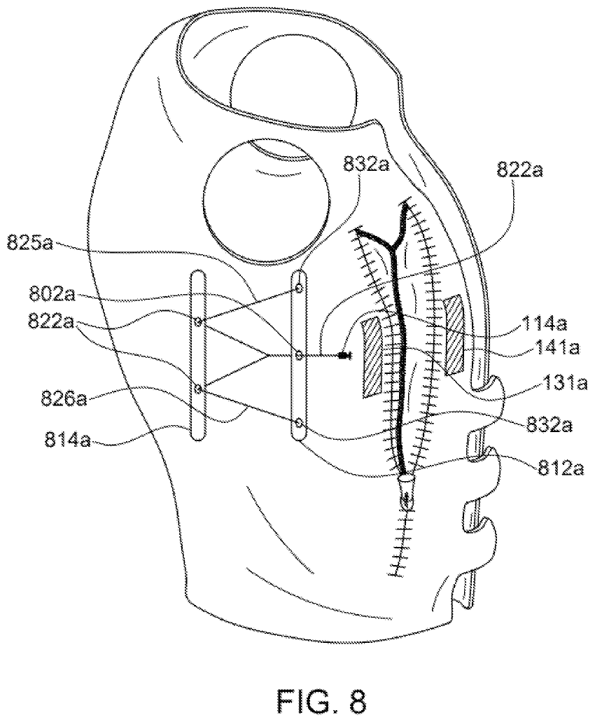

FIG. 8 depicts a side exterior view of one embodiment of a garment. In this embodiment, strips 812a and 814a are substantially parallel and lie along vertical portions of garment 100. Strip 812a has hole 802a which allows string 822a to pass through, and subsequently split into 2 substantially equal strings at point 113a, which continue to pass through strip 814a through grommets 822a and back in the opposite direction, connecting to strip 812a at points 832a. In the present embodiment, strings 826a and 825a are able to be attached and re-attached to points 832a, thereby enabling the complete de-lacing of the system, for example, to make it easier to machine wash the device. When String 822a is pulled inwards to the center of the jacket, strips 812a and 814a move towards each other, thereby bringing the vertical section of the garment they are attached to with them, and compressing the user.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by this disclosure. It is indeed intended that the scope of this disclosure should be determined by a proper legal interpretation and construction of the disclosure, including equivalents, as understood by those of skill in the art relying upon the complete disclosure present at the time of filing.

Use of these equivalent options presents equal derivative claim standing per the disclosed utility improvements disclosed regardless of specific technique elected.

What is claimed is:

1. A wearable pressure-applying device comprising of:
a number of mechanisms on said device adapted to secure said device to a user; and
a mechanism that can be manually or mechanically engaged that constricts device, applying pressure to the user.

2. The wearable pressure-applying device in claim 1, wherein a number of the mechanisms adapted to secure the device to the user applies an initial pressure that can be easily set by the user.

3. The wearable pressure-applying device in claim 1, wherein one or more of the closure mechanisms are zippers.

4. The wearable pressure-applying device in claim 1, wherein one or more of the closure mechanisms does not span the entirety of any vertical portion of the wearable device.

5. The wearable pressure-applying device in claim 1, wherein the mechanism adapted to constrict the wearable device uses threads laced through the wearable device across a substantial vertical section of the wearable device.

6. The wearable pressure-applying device in claim 1, wherein the mechanism adapted to constrict the wearable device uses strings laced through opposite-facing sides of the wearable device, as to bring both sides into each other when the string is pulled.

7. The wearable pressure-applying device in claims 5 and 6, wherein the connection between the strings and the wearable device is interrupted by separable connections to enable the user to attach and remove this mechanism.

8. The wearable pressure-applying device in claim 1, with an ergonomically contoured handle.

9. The wearable pressure-applying device in claim 8, wherein the handle can be easily and quickly attached to and removed from the mechanism adapted to constrict the device through the use of a coupler.

10. The wearable pressure-applying device in claim 1, that uses a cord lock mechanism to allow the continuance of the constriction of the wearable device to substantially the exact force the user initially applied.

11. The wearable pressure-applying device in claim 10,, with a quick release mechanism that will release the string, un-constricting the device, thereby releasing all the pressure that was being applied.

12. The wearable pressure-applying device in claim 11, wherein the cord-lock mechanism can be modularly attached or removed from the wearable device.

13. The wearable pressure-applying device in claim 1, with a mechanical mechanism that can be quickly attached to and removed from the mechanism adapted to constrict the device through the use of a coupler.

14. The wearable pressure-applying device in claim 13, wherein the mechanical mechanism can be modularly attached or removed from the wearable device.

15. The wearable pressure-applying device in claim 14, wherein the mechanical mechanism includes a quick release mechanism, which releases the string, un-constricting the device, thereby releasing all the pressure that was being applied.

16. The wearable pressure-applying device in claim 1, wherein the mechanical mechanism uses a battery.

17. The wearable pressure-applying device in claim 1, wherein the mechanical mechanism uses a number of sensors and can be enabled to determine when to apply pressure using data from said sensors.

18. The wearable pressure-applying device in claim 1, wherein the mechanical mechanism can be controlled wirelessly.
